(19) United States
(12) Patent Application Publication
(10) Pub. No.: US 2025/0268457 A1
(43) Pub. Date: Aug. 28, 2025

(54) INTEGRATIONS OF SENSING ELEMENTS AND ACCESS LUMENS FOR ENDOSCOPES

(71) Applicant: Auris Health, Inc., Santa Clara, CA (US)

(72) Inventors: Taylor Eric Furtado, Campbell, CA (US); Deena Jamal Malaeb, San Ramon, CA (US); Justin Gilbert, Santa Clara, CA (US); William Lanphier Chapin, Redwood City, CA (US)

(73) Assignee: Auris Health, Inc., Santa Clara, CA (US)

(21) Appl. No.: 19/060,385

(22) Filed: Feb. 21, 2025

Related U.S. Application Data
(60) Provisional application No. 63/557,453, filed on Feb. 23, 2024.

(51) Int. Cl. A61B 1/00; A61B 1/005; A61B 1/018

(52) U.S. Cl. CPC A61B 1/00114; A61B 1/00097; A61B 1/0011; A61B 1/00119; A61B 1/005; A61B 1/018

(57) ABSTRACT

This disclosure provides methods, devices, and systems for integrating sensing elements on medical instruments. The present implementations more specifically relate to techniques for disposing sensor loads (such as electrical wires) along the length of an elongate shaft in a way that reduces wear and tear (or fatigue) on the sensor loads during articulation of the shaft. In some aspects, the design of an endoscope or working channel can be improved by wrapping the sensor load in a helix, or helical configuration, along the length of the outer diameter of the working channel or the inner diameter of the endoscope. The helical configuration distributes the electrical wires more evenly around the circumference of the working channel or endoscope, thereby balancing the forces (such as tensile and compressive forces) imparted on the wires during bending or articulation of the shaft.

### CROSS-REFERENCE TO RELATED APPLICATIONS

This application claims priority and benefit under 35 U.S.C. § 119 (e) to U.S. Provisional Patent Application No. 63/557,453, filed Feb. 23, 2024, which is incorporated herein by reference in its entirety.

### TECHNICAL FIELD

This disclosure relates generally to medical robotics, and specifically to integrations of sensing elements and access lumens for robotic endoscopes.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside a patient's body. For example, in a PCNL procedure, a physician can insert a ureteroscope into the urinary tract through the urethra. A ureteroscope includes an endoscope at its distal end configured to enable visualization of the urinary tract. The physician navigates the ureteroscope through the bladder, up the ureter, and into the kidney where a kidney stone is located. Once at the site of a kidney stone (such as within a calyx of the kidney), the ureteroscope can be used to designate or “tag” a target location for a second medical instrument (such as a needle) to access the kidney percutaneously. The physician drives the needle into the patient, through the target location, and uses another medical instrument (which may be in conjunction with the needle) to extract the kidney stone from the patient via the percutaneous access point. The success or failure of a percutaneous medical procedure often depends on various factors, including the physician's skill, the patient's anatomy, as well as the quality and robustness of any tools or equipment the physician uses to perform the procedure.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a medical instrument including an elongate shaft, one or more sensors coupled to the elongate shaft, and a sensor load coupled to the one or more sensors and disposed in a helical configuration along a length of the elongate shaft. The sensor load is configured to carry electrical signals to or from the one or more sensors (such as to communicate sensor information with a control system).

Another innovative aspect of the subject matter of this disclosure can be implemented in a method for assembling a medical instrument. The method includes steps of attaching one or more sensors to an elongate shaft and disposing a sensor load in a helical configuration along a length of the elongate shaft so that the sensor load is coupled to the one or more sensors. The sensor load is configured to carry electrical signals to or from the one or more sensors (such as to communication sensor information with a control system).

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows a top view of the medical system of FIG. 1 configured to assist in inserting a scope into an anatomy.

FIG. 3 shows a top view of the medical system of FIG. 1 configured to navigate a scope within an anatomy.

FIG. 4 shows a top view of medical system of FIG. 1 configured to assist in inserting a needle into an anatomy.

FIGS. 5A and 5B show an example medical instrument, according to some implementations.

FIGS. 6A and 6B show an example system for helixing electrical wires around a working channel, according to some implementations.

FIG. 6C show another example system for helixing electrical wires around a working channel, according to some implementations.

FIG. 7A shows an example system and method for helixing electric wires around an inner diameter of an endoscope, according to some implementations.

FIG. 7B shows another example system and method for helixing electric wires around an inner diameter of an endoscope, according to some implementations.

FIGS. 8A-8D show an example endoscope having sensor elements integrated into its walls, according to some implementations.

FIG. 9 shows a cross-section of an endoscope, according to some implementations.

FIG. 10 shows an illustrative flowchart depicting an example operation for assembling a medical instrument, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

Many medical instruments used in robotically assisted medical procedures include various sensors (such as electromagnetic sensors) that can be used for positioning or navigating the instruments inside an anatomy. For example, a working channel carrying various sensors may be inserted down a lumen (or inner diameter) of an endoscope. The sensors are electrically coupled to a control system via electrical wires that share the same lumen as the working channel. In existing implementations, the electrical wires and the working channel are loaded into the lumen together but move independently of one another. As a result, the electrical wires and the working channel are often disposed on opposite sides of the endoscope's inner diameter. This design may impart unbalanced mechanical loads on the electrical wires based on the direction in which the shaft articulates. In some directions of articulation, the electrical wires may experience compressive loading (or compression). In some other directions of articulation, the electrical wires may experience tensile loading (or tension). Repeated flexing with high amounts of articulation may cause excessive wear and tear (or fatigue) on the electrical wires.

Further, depending on the number of sensors coupled to the working channel, multiple electrical wires may be disposed down the length of the endoscope. Such wires are often fused together in a ribbon configuration (also referred to as an “electrical ribbon”). Electrical ribbons have uneven flexural stiffness. For example, a ribbon is much stiffer bending in the “long” axis compared to bending in the “short” axis. Such uneven distribution of stiffness further complicates the task of tuning a robotically controlled endoscope. The increased stiffness in the long axis also causes reaction forces in the electrical ribbon to take the path of least resistance, which may impart further fatigue into the electrical wires. Aspects of the present disclosure recognize that the design of an endoscope or working channel can be improved by wrapping the sensor wires (or electrical ribbon) in a helix, or helical configuration, along the length of the outer diameter or the inner diameter of an elongate shaft (such as a working channel or an endoscope). The helical configuration distributes the electrical wires more evenly around the circumference of the working channel or endoscope, thereby balancing the forces imparted on the wires during any articulation of the elongate shaft.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope and/or a percutaneous access needle) to access a target site within an anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as a percutaneous access instrument that is exclusively controlled and operated by a physician). The systems and techniques described herein also may be applicable beyond the context of medical procedures (such as in simulated environments or laboratory settings, such as with models or simulators, among other examples).

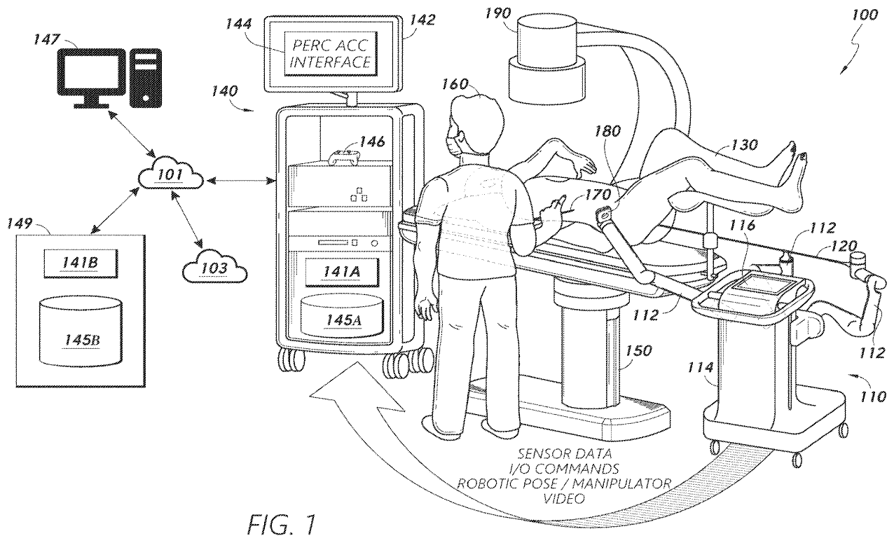

FIG. 1 shows an example medical system 100, according to some implementations. The medical system 100 includes a robotic system 110 configured to engage with or control a medical instrument to perform a procedure on a patient 130. The medical system 100 also includes a control system 140 configured to interface with the robotic system 110, provide information regarding the procedure, or perform a variety of other operations. For example, the control system 140 can include a display(s) 142 to present certain information to assist the physician 160. The display(s) 142 may be a monitor, screen, television, virtual reality hardware, augmented reality hardware, or three-dimensional imaging devices (such as hologram devices), among other examples, or combinations thereof. The medical system 100 can include a table 150 configured to hold the patient 130. The system 100 can further include an electromagnetic (EM) field generator 180, which can be held by one or more robotic arms 112 of the robotic system 110 or can be a stand-alone device. In examples, the medical system 100 can also include an imaging device 190 which can be integrated into a C-arm or configured to provide imaging during a procedure, such as for a fluoroscopy-type procedure. Although shown in FIG. 1, in some implementations the imaging device 190 is eliminated.

In some implementations, the medical system 100 can be used to perform a percutaneous procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate a first medical instrument (such as a scope) from the urethra, through the bladder, up the ureter, and into the kidney where the stone is located. The control system 140 can provide information via the display(s) 142 regarding the first medical instrument to assist the physician 160 in navigating the first medical instrument, such as real-time images captured therewith.

Once at the site of the kidney stone (such as within a calyx of the kidney), the first medical instrument can be used to designate or tag a target location for a second medical instrument (such as a needle) to access the kidney percutaneously (such as a desired point to access the kidney). To minimize damage to the kidney or the surrounding anatomy, the physician 160 can designate a particular papilla as the target location for entering into the kidney with the second medical instrument. However, other target locations can be designated or determined. To assist the physician in driving the second medical instrument into the patient 130 through the particular papilla, the control system 140 can provide a percutaneous access interface 144, which can include a visualization to indicate an alignment of an orientation of the second medical instrument relative to a target trajectory (such as a desired access path from the patient's skin to the target location), a visualization to indicate a progress of inserting the second medical instrument into the kidney towards the target location, guidance on the percutaneous procedure, or other information. Once the second medical instrument has reached the target location (as determined, such as by sensors attached to the needle 170, the scope 120, or other any sensor or imaging modality), the physician 160 can use the second medical instrument or another medical instrument to extract the kidney stone from the patient 130, such as through the percutaneous access point.

Although the above percutaneous procedure or other procedures are discussed in the context of using the first medical instrument, in some implementations a percutaneous procedure can be performed without the assistance of the first medical instrument. Further, the medical system 100 can be used to perform a variety of other procedures. Moreover, although many implementations describe the physician 160 using the second medical instrument, the second medical instrument can alternatively be used by a component of the medical system 100. For example, the second medical instrument can be held or manipulated by the robotic system 110 (such as the one or more robotic arms 112) and the techniques discussed herein can be implemented to control the robotic system 110 to insert the second medical instrument with the appropriate pose (or aspect of a pose, such as orientation or position) to reach a target location.

In the example of FIG. 1, the first medical instrument is implemented as a scope 120 and the second medical instrument is implemented as a needle 170. Thus, for ease of discussion, the first medical instrument is referred to as “the scope 120” or “the lumen-based medical instrument,” and the second medical instrument is referred to as “the needle 170” or “the percutaneous medical instrument.” However, the first medical instrument and the second medical instrument can each be implemented as a suitable type of medical instrument including, for example, a scope (sometimes referred to as an “endoscope”), a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction or irrigation tool, or clip applier, among other examples. In some implementations, a medical instrument is a steerable device, while some other implementations a medical instrument is a non-steerable device. In some implementations, a surgical tool refers to a device that is configured to puncture or to be inserted through the human anatomy, such as a needle, a scalpel, or a guidewire, among other examples. However, a surgical tool can refer to other types of medical instruments.

In some implementations, a medical instrument, such as the scope 120 or the needle 170, includes a sensor that is configured to generate sensor data, which can be sent to another device. In examples, sensor data can indicate a location or orientation of the medical instrument or can be used to determine a location or orientation of the medical instrument. For instance, a sensor can include an electromagnetic (EM) sensor with a coil of conductive material. Here, an EM field generator, such as the EM field generator 180, can provide an EM field that is detected by the EM sensor on the medical instrument. The magnetic field can induce small currents in coils of the EM sensor, which can be analyzed to determine a distance or angle or orientation between the EM sensor and the EM field generator. Further, a medical instrument can include other types of sensors configured to generate sensor data, such as one or more of any of: a camera, a range sensor, a radar device, a shape sensing fiber, an accelerometer, a gyroscope, a satellite-based positioning sensor (such as a global positioning system (GPS)), or a radio-frequency transceiver, among other examples. In some implementations, a sensor is positioned on a distal end of a medical instrument, while in some other implementations a sensor is positioned at another location on the medical instrument. In some implementations, a sensor on a medical instrument can provide sensor data to the control system 140 and the control system 140 can perform one or more localization techniques to determine or track a position or an orientation of a medical instrument.

In some implementations, the medical system 100 may record or otherwise track the runtime data that is generated during a medical procedure. For example, the medical system 100 may track or otherwise record the sensor readings (such as sensor data) from the instruments (such as the scope 120 and the needle 170) in case data store 145A (such as a computer storage system, such as computer readable memory, database, or filesystem, among other examples). In addition to sensor data, the medical system 100 can store other types of case logs in the case data store 145A. For example, in the context of FIG. 1, the case logs can include time series data of the video images captured by the scope 120, status of the robotic system 110, commanded data from an I/O device(s) (such as I/O device(s) 146), audio data (such as may be captured by audio capturing devices embedded in the medical system 100, such as microphones on the medical instruments, robotic arms, or elsewhere in the medical system), external (relative to the patient) imaging device (such as RGB cameras, LIDAR imaging sensors, or fluoroscope imaging sensors), among other examples.

As shown in FIG. 1, the control system 140 includes an analytics engine 141A which may operate on the case logs stored in the case data store 145A to label the case logs according to a procedure phase for a given time. In some implementations, the analytics engine 141A may employ machine learning techniques to segment the medical procedure according to the different phases. In some implementations, once the medical procedure has been segmented, the analytics engine 141A may generate metrics for the medical procedure phases, the medical procedure generally, or provide insights and recommendations to the users of the medical system 100 (such as physicians, staff, or training personnel).

FIG. 1 further shows that in some implementations the control system 140 may include a network connection (such as via network 101) to a cloud-based data analytics platform 149. The cloud-based data analytics platform 149 may be a computer system that provides third party computers 147 postoperative analytic capabilities on a given medical procedure or analytics across multiple medical procedures. As shown in FIG. 1, the cloud-based data analytics platform 149 may further connect to additional medical systems 103, which each in turn may transmit case logs to the cloud-based data analytics platform 149. Because the cloud-based data analytics platform receives case logs from multiple medical systems, the cloud-based data analytics platform 149 may have access to a comparatively larger pool of data than a single medical system would have access to and may in turn aggregate the case logs across multiple medical systems to derive medical procedure insights. Medical procedure insights may include guidance on factors that result in an increased likelihood for success in a medical procedure based on the metrics derived from segmenting the case logs across medical systems and across medical procedures.

As shown in FIG. 1, the cloud-based data analytics platform 149 may include an analytics engine 141B and cloud-based data store 145B. The cloud-based data store 145B may be a computer storage device that stores the system data records from the medical system 100 and the additional medical systems 103. The analytics engine 141B may include features and capabilities similar to the analytics engine 141A. However, in some implementations, the analytics engine 141A may further operate to analyze case logs across multiple medical systems (such as medical system 100 and medical systems 103) to generate metrics or insights. This may provide comparatively robust insights because the data used to generate such metrics or insights is using a broader range of information. Additionally, or alternatively, the cloud-based analytics engine 141B may uses machine learning techniques that are suitable for post-operative classification, whereas the local analytics engine 141B may use machine learning techniques that are suitable for real-time or near-real time classification.

The term “scope” or “endoscope” is used herein according to its broad and ordinary meanings and can refer to any type of elongate medical instrument having image generating, viewing, or capturing functionality and configured to be introduced into any type of organ, cavity, lumen, chamber, or space of a body. For example, references herein to scopes or endoscopes can refer to a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), or a borescope, among other examples.

A scope can comprise a tubular or flexible medical instrument that is configured to be inserted into the anatomy. In some implementations, a scope can accommodate wires or optical fibers to transfer signals to or from an optical assembly and a distal end of the scope, which can include an imaging device, such as an optical camera. The camera or imaging device can be used to capture images of an internal anatomical space, such as a target calyx or papilla of a kidney. A scope can further be configured to accommodate optical fibers to carry light from proximately-located light sources, such as light-emitting diodes, to the distal end of the scope. The distal end of the scope can include ports for light sources to illuminate an anatomical space when using the camera or imaging device. In some implementations, the scope is configured to be controlled by a robotic system, such as the robotic system 110. The imaging device can comprise an optical fiber, fiber array, or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some implementations, a scope is configured to be articulated with, for example, five or six degrees of freedom, including X, Y, Z coordinate movement, as well as pitch, yaw, and roll. A position sensor(s) of the scope can likewise have similar degrees of freedom with respect to the position information they produce or provide. A scope can include telescoping parts, such as an inner leader portion and an outer sheath portion, which can be manipulated to telescopically extend the scope. A scope, in some instances, can comprise a rigid or flexible tube, and can be dimensioned to be passed within an outer sheath, catheter, introducer, or other lumen-type device, or can be used without such devices. In some implementations, a scope includes a working channel for deploying medical instruments (such as lithotripters, basketing devices, or forceps), irrigation, or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with or control the scope 120 to perform a procedure. As shown, each robotic arm 112 can include multiple arm segments coupled to joints, which can provide multiple degrees of movement. In the example of FIG. 1, the robotic system 110 is positioned proximate to the patient's 130 legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 can also be connected to the EM field generator 180, which can be positioned near a treatment site, such as within proximity to the kidneys of the patient 130.

The robotic system 110 can also include a support structure 114 coupled to the one or more robotic arms 112. The support structure 114 can include control electronics or circuitry, one or more power sources, one or more pneumatics, one or more optical sources, one or more actuators (such as motors to move the one or more robotic arms 112), memory or data storage, or one or more communication interfaces. In some implementations, the support structure 114 includes an input/output (I/O) device(s) 116 configured to receive input, such as user input to control the robotic system 110, or provide output, such as a graphical user interface (GUI), information regarding the robotic system 110, or information regarding a procedure, among other examples. The I/O device(s) 116 can include a display, a touchscreen, a touchpad, a projector, a mouse, a keyboard, a microphone, or a speaker. In some implementations, the robotic system 110 is movable (such as the support structure 114 includes wheels) so that the robotic system 110 can be positioned in a location that is appropriate or desired for a procedure. In some other implementations, the robotic system 110 is a stationary system. Further, in some implementations, the robotic system 110 is integrated into the table 150.

The robotic system 110 can be coupled to any component of the medical system 100, such as the control system 140, the table 150, the EM field generator 180, the scope 120, or the needle 170. In some implementations, the robotic system is communicatively coupled to the control system 140. In one example, the robotic system 110 can be configured to receive a control signal from the control system 140 to perform an operation, such as to position a robotic arm 112 in a particular manner, or manipulate the scope 120, among other examples. In response, the robotic system 110 can control a component of the robotic system 110 to perform the operation. In another example, the robotic system 110 is configured to receive an image from the scope 120 depicting internal anatomy of the patient 130 or send the image to the control system 140, which can then be displayed on the display(s) 142. Furthermore, in some implementations, the robotic system 110 is coupled to a component of the medical system 100, such as the control system 140, in such a manner as to allow for fluids, optics, or power, among other examples, to be received therefrom.

The control system 140 can be configured to provide various functionality to assist in performing a medical procedure. In some implementations, the control system 140 can be coupled to the robotic system 110 and operate in cooperation with the robotic system 110 to perform a medical procedure on the patient 130. For example, the control system 140 can communicate with the robotic system 110 via a wireless or wired connection (such as to control the robotic system 110 or the scope 120, receive an image(s) captured by the scope 120), provide fluids to the robotic system 110 via one or more fluid channels, provide power to the robotic system 110 via one or more electrical connections, provide optics to the robotic system 110 via one or more optical fibers or other components, among other examples. Further, in some implementations, the control system 140 can communicate with the needle 170 or the scope 120 to receive sensor data from the needle 170 or the scope 120 (via the robotic system 110 or directly from the needle 170 or the scope 120). Moreover, in some implementations, the control system 140 can communicate with the table 150 to position the table 150 in a particular orientation or otherwise control the table 150. Further, in some implementations, the control system 140 can communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, a keyboard, a surgeon or physician console, virtual reality hardware, augmented hardware, microphone, speakers, or haptic devices, among other examples.

As also shown in FIG. 1, the control system 140 can include the display(s) 142 to provide various information regarding a procedure. The display(s) 142 can present the percutaneous access interface 144 to assist the physician 160 in the percutaneous access procedure (such as manipulating the needle 170 towards a target site). The display(s) 142 can also provide (such as via the percutaneous access interface 144 or another interface) information regarding the scope 120. For example, the control system 140 can receive real-time images that are captured by the scope 120 and display the real-time images via the display(s) 142. Additionally, or alternatively, the control system 140 can receive signals (such as analog, digital, electrical, acoustic or sonic, pneumatic, tactile, hydraulic) from a medical monitor or a sensor associated with the patient 130, and the display(s) 142 can present information regarding the health or environment of the patient 130. Such information can include information that is displayed via a medical monitor including, for example, a heart rate (such as ECG or HRV), blood pressure or rate, muscle bio-signals (such as EMG), body temperature, blood oxygen saturation (such as SpO2), CO2, brainwaves (such as EEG), environmental or local or core body temperature, among other examples.

To facilitate the functionality of the control system 140, the control system 140 can include various components (sometimes referred to as “subsystems”). For example, the control system 140 can include control electronics or circuitry, as well as one or more power sources, pneumatics, optical sources, actuators, memory or data storage devices, or communication interfaces. In some implementations, the control system 140 includes control circuitry comprising a computer-based control system that is configured to store executable instructions, that when executed, cause various operations to be implemented. In some implementations, the control system 140 is movable, such as that shown in FIG. 1, while in some other implementations, the control system 140 is a stationary system. Although various functionality and components are discussed as being implemented by the control system 140, any of this functionality or components can be integrated into or performed by other systems or devices, such as the robotic system 110, the table 150, or the EM field generator 180 (or even the scope 120 or the needle 170).

The imaging device 190 can be configured to capture or generate one or more images of the patient 130 during a procedure, such as one or more x-ray or CT images. In examples, images from the imaging device 190 can be provided in real-time to view anatomy or medical instruments, such as the scope 120 or the needle 170, within the patient 130 to assist the physician 160 in performing a procedure. The imaging device 190 can be used to perform a fluoroscopy (such as with a contrast dye within the patient 130) or another type of imaging technique. Although shown in FIG. 1, in many implementations the imaging device 190 is not implemented for performing a procedure or the imaging device 190 (including the C-arm) is eliminated.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, or the Internet. Further, in some implementations, the components of the medical system 100 are connected for data communication, fluid or gas exchange, or power exchange, among other examples, via one or more support cables, or tubes, among other examples.

The medical system 100 can provide a variety of benefits, such as providing guidance to assist a physician in performing a procedure (such as instrument tracking or instrument alignment information), enabling a physician to perform a procedure from an ergonomic position without the need for awkward arm motions or positions, enabling a single physician to perform a procedure with one or more medical instruments, avoiding radiation exposure (such as associated with fluoroscopy techniques), enabling a procedure to be performed in a single-operative setting, or providing continuous suction to remove an object more efficiently (such as to remove a kidney stone), among other examples. For example, the medical system 100 can provide guidance information to assist a physician in using various medical instruments to access a target anatomical feature while minimizing bleeding or damage to anatomy (such as critical organs or blood vessels). Further, the medical system 100 can provide non-radiation-based navigational or localization techniques to reduce physician and patient exposure to radiation or reduce the amount of equipment in the operating room. Moreover, the medical system 100 can provide functionality that is distributed between at least the control system 140 and the robotic system 110, which can be independently movable. Such distribution of functionality or mobility can enable the control system 140 or the robotic system 110 to be placed at locations that are optimal for a particular medical procedure, which can maximize working area around the patient or provide an optimized location for a physician to perform a procedure.

Although various techniques and systems are discussed as being implemented as robotically-assisted procedures (such as procedures that at least partly use the medical system 100), the techniques and systems can be implemented in other procedures, such as in fully-robotic medical procedures, or human-only procedures (such as free of robotic systems), among other examples. For example, the medical system 100 can be used to perform a procedure without a physician holding or manipulating a medical instrument (such as a fully-robotic procedure). That is, medical instruments that are used during a procedure, such as the scope 120 and the needle 170, can each be held or controlled by components of the medical system 100, such as the robotic arm(s) 112 of the robotic system 110.

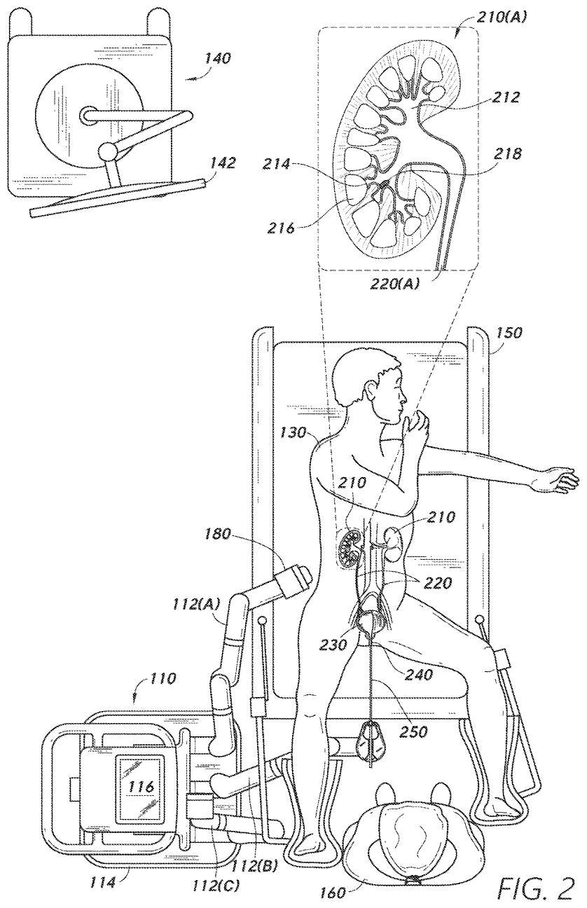

FIGS. 2-4 illustrate a top view of the medical system 100 of FIG. 1 arranged to perform a percutaneous procedure in accordance with one or more implementations. In these examples, the medical system 100 is arranged in an operating room to remove a kidney stone from the patient 130 with the assistance of the scope 120 and the needle 170. In some implementations of such a procedure, the patient 130 is positioned in a modified supine position with the patient 130 slightly tilted to the side to access the flank of the patient 130, such as that illustrated in FIG. 1. However, the patient 130 can be positioned in other manners, such as a supine position, or a prone position, among other examples. For ease of illustration in viewing the anatomy of the patient 130, FIG. 2-4 illustrate the patient 130 in a supine position with the legs spread apart. Also, for ease of illustration, the imaging device 190 (including the C-arm) has been removed.

Although FIGS. 2-4 illustrate use of the medical system 100 to perform a percutaneous procedure to remove a kidney stone from the patient 130, the medical system 100 can be used to remove a kidney stone in other manners or to perform other procedures. Further, the patient 130 can be arranged in other positions as desired for a procedure. Various acts are described in FIGS. 2-4 and throughout this disclosure as being performed by the physician 160. It should be understood that these acts can be performed directly by the physician 160, a user under direction of the physician, another user (such as a technician), a combination thereof, or any other user.

FIGS. 2-4 show various features of the anatomy of the patient 130. For example, the patient 130 includes kidneys 210 fluidly connected to a bladder 230 via ureters 220, and a urethra 240 fluidly connected to the bladder 230. As shown in the enlarged depiction of the kidney 210(A), the kidney 210(A) includes calyces (including calyx 212), renal papillae (including the renal papilla 214, also referred to as “the papilla 214”), and renal pyramids (including the renal pyramid 216). In these examples, a kidney stone 218 is located in proximity to the papilla 214. However, the kidney stone 218 can be located at other locations within the kidney 210(A) or elsewhere.

As shown in FIG. 2, to remove the kidney stone 218 in the example percutaneous procedure, the physician 160 can position the robotic system 110 at the side or foot of the table 150 to initiate delivery of the scope 120 (not illustrated in FIG. 2) into the patient 130. In particular, the robotic system 110 can be positioned at the side of the table 150 within proximity to the feet of the patient 130 and aligned for direct linear access to the urethra 240 of the patient 130. In examples, the hip of the patient 130 is used as a reference point to position the robotic system 110. Once positioned, one or more of the robotic arms 112, such as the robotic arms 112(B) and 112(C), can stretch outwards to reach in between the legs of the patient 130. For example, the robotic arm 112(B) can be controlled to extend and provide linear access to the urethra 240, as shown in FIG. 2. In this example, the physician 160 inserts a medical instrument 250 at least partially into the urethra 240 along this direct linear access path (sometimes referred to as “a virtual rail”). The medical instrument 250 can include a lumen-type device configured to receive the scope 120, thereby assisting in inserting the scope 120 into the anatomy of the patient 130. By aligning the robotic arm 112(B) to the urethra 240 of the patient 130 or using the medical instrument 250, friction or forces on the sensitive anatomy in the area can be reduced. Although the medical instrument 250 is illustrated in FIG. 2, in some implementations, the medical instrument 250 is not used (such as the scope 120 can be inserted directly into the urethra 240).

The physician 160 can also position the robotic arm 112(A) near a treatment site for the procedure. For example, the robotic arm 112(A) can be positioned within proximity to the incision site or the kidneys 210 of the patient 130. The robotic arm 112(A) can be connected to the EM field generator 180 to assist in tracking a location of the scope 120 or the needle 170 during the procedure. Although the robotic arm 112(A) is positioned relatively close to the patient 130, in some implementations the robotic arm 112(A) is positioned elsewhere or the EM field generator 180 is integrated into the table 150 (which can allow the robotic arm 112(A) to be in a docked position). In this example, at this point in the procedure, the robotic arm 112(C) remains in a docked position, as shown in FIG. 2. However, the robotic arm 112(C) can be used in some implementations to perform any of the functions of the robotic arms 112(A) or 112(C).

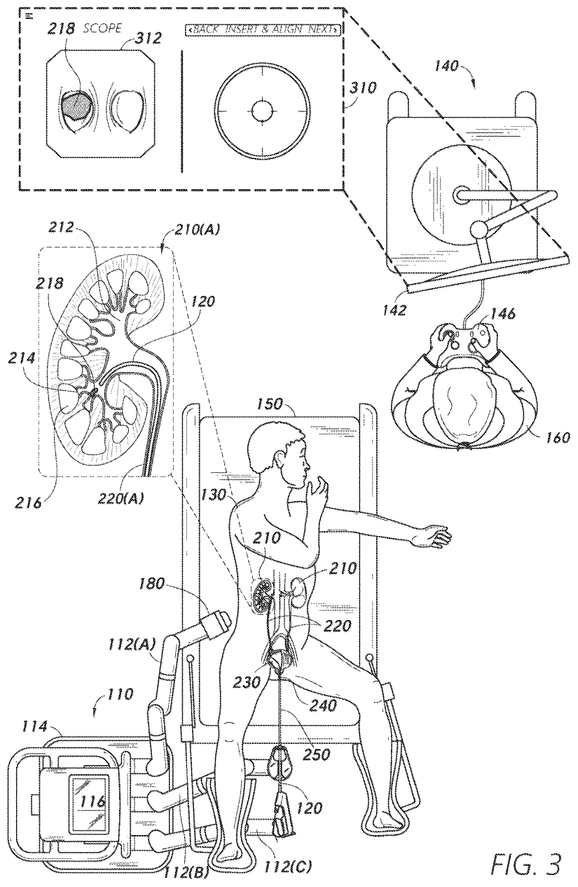

Once the robotic system 110 is properly positioned or the medical instrument 250 is inserted at least partially into the urethra 240, the scope 120 can be inserted into the patient 130 robotically, manually, or a combination thereof, as shown in FIG. 3. For example, the physician 160 can connect the scope 120 to the robotic arm 112(C) or position the scope 120 at least partially within the medical instrument 250 or the patient 130. The scope 120 can be connected to the robotic arm 112(C) at any time, such as before the procedure or during the procedure (such as after positioning the robotic system 110). The physician 160 can then interact with the control system 140, such as the I/O device(s) 146, to navigate the scope 120 within the patient 130. For example, the physician 160 can provide input via the I/O device(s) 146 to control the robotic arm 112(C) to navigate the scope 120 through the urethra 240, the bladder 230, the ureter 220(A), and up to the kidney 210(A).

As shown, the control system 140 can present an instrument-alignment interface 310, such as the instrument-alignment interface 310 of FIG. 3, via the display(s) 142 to view a real-time image 312 captured by the scope 120 to assist the physician 160 in controlling the scope 120. The physician 160 can navigate the scope 120 to locate the kidney stone 218, as depicted in the image 312. In some implementation, the control system 140 can use localization techniques to determine a position or an orientation of the scope 120, which can be viewed by the physician 160 through the display(s) 142 (not illustrated on the display(s) 142 in FIG. 3) to also assist in controlling the scope 120. Further, in some implementations, other types of information can be presented through the display(s) 142 to assist the physician 160 in controlling the scope 120, such as x-ray images of the internal anatomy of the patient 130.

Upon locating the kidney stone 218, the physician 160 can identify a location for the needle 170 to enter the kidney 210(A) for eventual extraction of the kidney stone 218. For example, to minimize bleeding or avoid hitting a blood vessel or other undesirable anatomy of the kidney 210(A) or anatomy surrounding the kidney 210(A), the physician 160 can seek to align the needle 170 with an axis of a calyx (such as can seek to reach the calyx head-on through the center of the calyx). To do so, the physician 160 can identify a papilla as a target location. In this example, the physician 160 uses the scope 120 to locate the papilla 214 that is near the kidney stone 218 and designate the papilla 214 as the target location. In some implementations of designating the papilla 214 as the target location, the physician can cause the medical system to tag the papilla. In tagging the papilla, the physician 160 can navigate the scope 120 to contact the papilla 214 and provide a UI input to the system to indicate the tagging, the control system 140 can use localization techniques to determine a location of the scope 120 (such as a location of the end of the scope 120), and the control system 140 can associate the location of the scope 120 with the target location. Additionally, or alternatively, the physician 160 can navigate the scope 120 to be within a particular distance to the papilla 214 (such as park in front of the papilla 214) and provide input indicating that the target location is within a field-of-view of the scope 120. The control system 140 can perform image analysis or other localization techniques to determine a location of the target location. In some other implementations, the scope 120 can deliver a fiduciary to mark the papilla 214 as the target location.

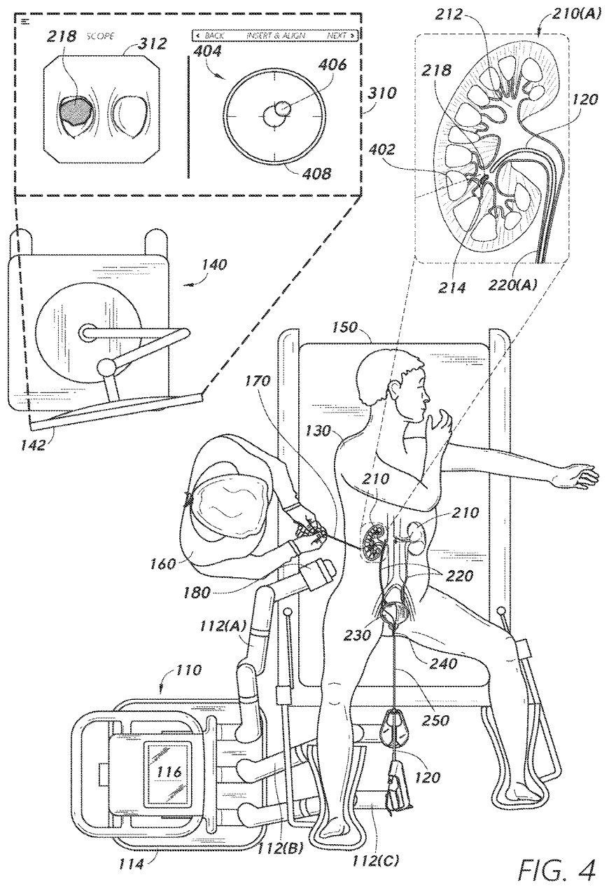

As shown in FIG. 4, the physician 160 can proceed with the procedure by positioning the needle 170 for insertion into the target location. In some implementations, the physician 160 can use his or her best judgment to place the needle 170 on the patient 130 at an incision site, such as based on knowledge regarding the anatomy of the patient 130, experience from previously performing the procedure, an analysis of CT or x-ray images or other pre-operative information of the patient 130, among other examples. Further, in some implementations, the control system 140 can provide information regarding a location to place the needle 170 on the patient 130. The physician 160 can attempt to avoid critical anatomy of the patient 130, such as the colon, paraspinal muscles, ribs, intercostal nerves, lungs, or pleura. In some examples, the control system 140 can use CT, x-ray, or ultrasound images to provide information regarding a location to place the needle 170 on the patient 130.

The control system 140 can determine a target trajectory 402 for inserting the needle 170 to assist the physician 160 in reaching the target location (such as the papilla 214). The target trajectory 402 can represent a desired path for accessing the target location. The target trajectory 402 can be determined based on a position of a medical instrument (such as the needle 170 or the scope 120), a target location within the human anatomy, a position or orientation of a patient, or the anatomy of the patient (such as the location of organs within the patient relative to the target location), among other examples. In this example, the target trajectory 402 includes a straight line that passes through the papilla 214 and the needle 170 (such as extends from a tip of the needle 170 through the papilla 214, such as a point on an axis of the papilla 214). However, the target trajectory 402 can take other forms, such as a curved line, or can be defined in other manners. In some examples, the needle 170 is implemented a flexible bevel-tip needle that is configured to curve as the needle 170 is inserted in a straight manner. Such needle can be used to steer around particular anatomy, such as the ribs or other anatomy. Here, the control system 140 can provide information to guide a user, such as to compensate for deviation in the needle trajectory or to maintain the user on the target trajectory.

Although the example of FIG. 4 illustrates the target trajectory 402 extending coaxially through the papilla 214, the target trajectory 402 can have another position, angle, or form. For example, a target trajectory can be implemented with a lower pole access point, such as through a papilla located below the kidney stone 218 shown in FIG. 4, with a non-coaxial angle through the papilla, which can be used to avoid the hip.

The control system 140 can use the target trajectory 402 to provide an alignment-progress visualization 404 via the instrument-alignment interface 310. For example, the alignment-progress visualization 404 can include an instrument alignment element 406 indicative of an orientation of the needle 170 relative to the target trajectory 402. The physician 160 can view the alignment-progress visualization 404 and orient the needle 170 to the appropriate orientation (such as the target trajectory 402). When aligned, the physician 160 can insert the needle 170 into the patient 130 to reach the target location. The alignment-progress visualization 404 can provide a progress visualization 408 (also referred to as “the progress bar 408”) indicative of a proximity of the needle 170 to the target location. As such, the instrument-alignment interface 310 can assist the physician 160 in aligning or inserting the needle 170 to reach the target location.

Once the target location has been reached with the needle 170, the physician 160 can insert another medical instrument, such as a power catheter, vacuum, or nephroscope into the path created by the needle 170 or over the needle 170. The physician 160 can use the other medical instrument or the scope 120 to fragment and remove pieces of the kidney stone 218 from the kidney 210(A).

In some implementations, a position of a medical instrument can be represented with a point, point set, or an orientation of the medical instrument can be represented as an angle or offset relative to an axis or plane. For example, a position of a medical instrument can be represented with a coordinate(s) of a point or point set within a coordinate system (such as one or more X, Y, Z coordinates) or an orientation of the medical instrument can be represented with an angle relative to an axis or plane for the coordinate system (such as angle with respect to the X-axis or plane, Y-axis or plane, or Z-axis or plane). Here, a change in orientation of the medical instrument can correspond to a change in an angle of the medical instrument relative to the axis or plane. Further, in some implementations, an orientation of a medical instrument is represented with yaw, pitch, or roll information. In some other implementations, an orientation of a medical instrument is represented in quaternion representation. Quaternion representation may avoid singularities present in a representation based on yaw, pitch, and roll.

In some implementations, a trajectory may refer to a pose. For example, a trajectory of a medical instrument can refer to a pose of the medical instrument, including or indicating both a position and orientation of the medical instrument. Similarly, a target trajectory can refer to a target pose, including or indicating both a position and orientation of a desired path. However, in some other implementations, a trajectory refers to either an orientation or a position.

Although particular robotic arms of the robotic system 110 are illustrated (or described herein) as performing particular functions in the context of FIGS. 2-4, any of the robotic arms 112 can be used to perform the functions. Further, any additional robotic arms or systems can be used to perform the procedure. Moreover, the robotic system 110 can be used to perform other parts of the procedure. For example, the robotic system 110 can be controlled to align or insert the needle into the patient 130. To illustrate, one of the robotic arms 112 can engage with or control the needle 170 to position the needle 170 at the appropriate location, align the needle 170 with the target trajectory, or insert the needle 170 to the target location. The control system 140 can use localization techniques to perform such processing. As such, in some implementations, a percutaneous procedure can be performed entirely or partially with the medical system 100 (such as with or without the assistance of the physician 160).

As described with reference to FIGS. 1-4, many medical instruments used in robotically assisted medical procedures include various sensors (such as EM sensors) that can be used for positioning or navigating the instruments inside an anatomy. For example, a working channel carrying various sensors may be inserted down a lumen (or inner diameter) of an endoscope (such as the scope 120). The sensors are electrically coupled to a control system (such as the control system 140) via electrical wires that share the same lumen as the working channel. In existing implementations, the electrical wires and the working channel are loaded into the lumen together but move independently of one another. Aspects of the present disclosure recognize that the design of an endoscope or working channel can be improved by wrapping the sensor wires (or electrical ribbon) in a helix, or helical configuration, along the length of the outer diameter or the inner diameter of an elongate shaft (such as a working channel or an endoscope). The helical configuration distributes the electrical wires more evenly around the circumference of the working channel or endoscope, thereby balancing the forces imparted on the wires during any articulation of the elongate shaft.

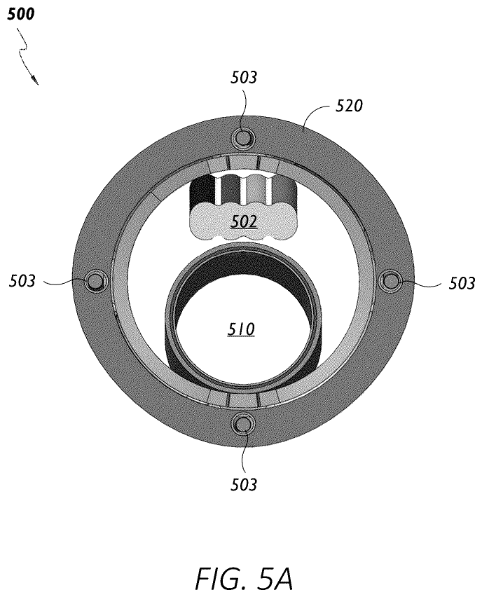

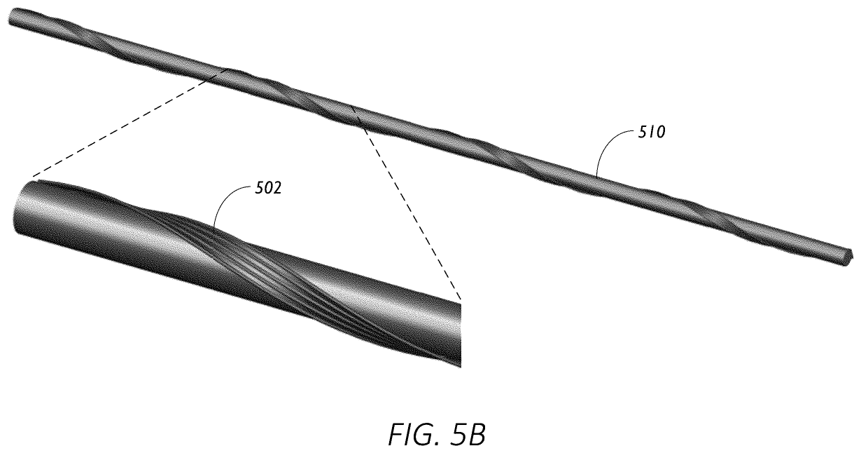

FIGS. 5A and 5B show an example medical instrument 500, according to some implementations. The medical instrument 500 includes an endoscope 520 having an inner diameter (or lumen) in which a working channel 510 and a sensor load 502 are disposed. FIG. 5A shows a cross-section of the medical instrument 500 and FIG. 5B shows an isometric view of the working channel 510 and the sensor load 502. In some implementations, the endoscope 520 may be one example of the scope 102 of FIGS. 1-4. The endoscope 520 includes a number of articulating mechanisms (also referred to as “tendons”) 503 that can be used to bend, deflect, or otherwise articulate the endoscope 520. As used herein, the term “sensor load” may refer to any electrical wires, circuitry, or sensor components associated with a sensing system. For simplicity of discussion, the sensor load 502 is described in the context of electrical wires. Thus, the terms “sensor load” and “electrical wires” may be used interchangeably herein. The electrical wires 502 may carry electrical signals to or from a set of sensors coupled to the working channel 510 (not shown for simplicity). In the example of FIG. 5B, the set of electrical wires 502 includes 5 conductors fused together as an electrical ribbon. However, in actual implementations, the electrical wires 502 may include fewer or more conductors than those depicted in FIG. 5B.

As shown in FIG. 5B, the electrical wires 502 are wrapped or wound in a helix down the length of the out diameter of the working channel 510. Compared to existing medical instruments in which electrical wires are loosely disposed along a single side of a working channel, the electrical wires 502 can be more tightly bound or coupled to the outer diameter of the working channel 510. This allows the medical instrument 500 to be more easily inserted into the lumen of an endoscope. Additionally, the helical configuration of the electrical wires 502 distributes forces more evenly along the length of the wires. For example, when the working channel 510 bends or articulates, a portion of each wire 502 may be disposed along an outer curvature of the bend and another portion of each wire 502 may be disposed along an inner curvature of the bend. The portion of the wire along the outer curvature is pulled by the working channel 510, under tensile loading, while the portion of the wire along the inner curvature is pushed by the working channel 510, under compressive loading. The tensile and compressive loads help offset one another to reduce the overall strain or fatigue on the electrical wires 502.

Aspects of the present disclosure recognize that some sections of the working channel 510 may be articulated (also referred to as “articulating sections”) while other sections of the working channel 510 merely bend or follow the articulating sections (also referred to as “passive sections”). Thus, the bend angle of the working channel 510 may vary across the length of the channel. To offset the forces on the electrical wires 502 during bending or articulation of the working channel 510, at least a portion of each wire should be disposed along the outer curvature of the bend and at least a portion of each wire should be disposed along the inner curvature of the bend (so that each of the electrical wires 502 experiences compressive loading and tensile loading). The twist rate or frequency of the helixes may depend on various factors including, but not limited to, the placement or alignment of sensors along the working channel 510 and/or the degree of bend or articulation of the working channel 510. As used herein, the terms “twist rate” or “frequency” refer to the number of helical rotations of the electrical wires 502 along a given length of the working channel 510. In some implementations, the twist rate of the helixes may be greater in more articulable sections of the working channel 510 and twist rate of the helixes may be lower in less articulable sections of the working channel 510. In some implementations, the twist rate of the helixes may be designed so that the compressive loading of the electrical wires 502 is substantially equal to the tensile loading of the electrical wires 502 throughout the bending or articulation of the working channel 510.

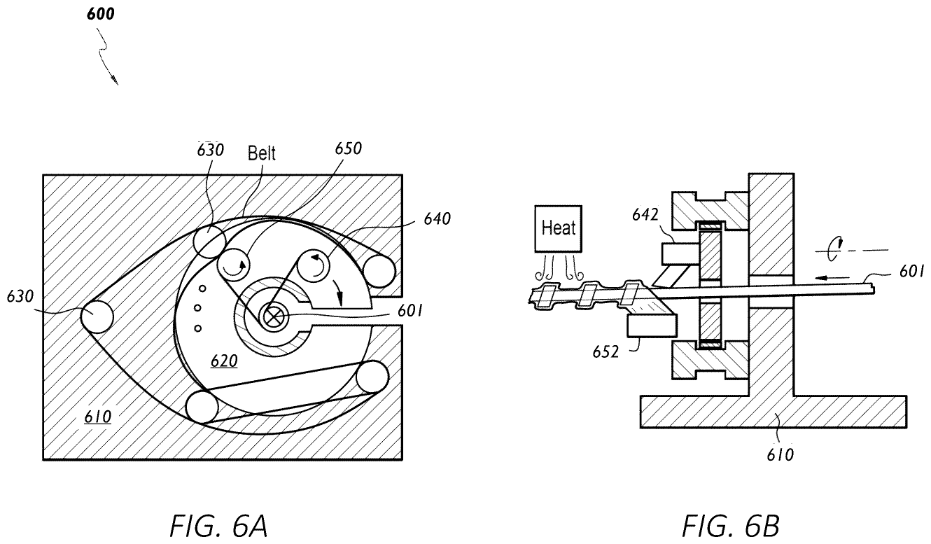

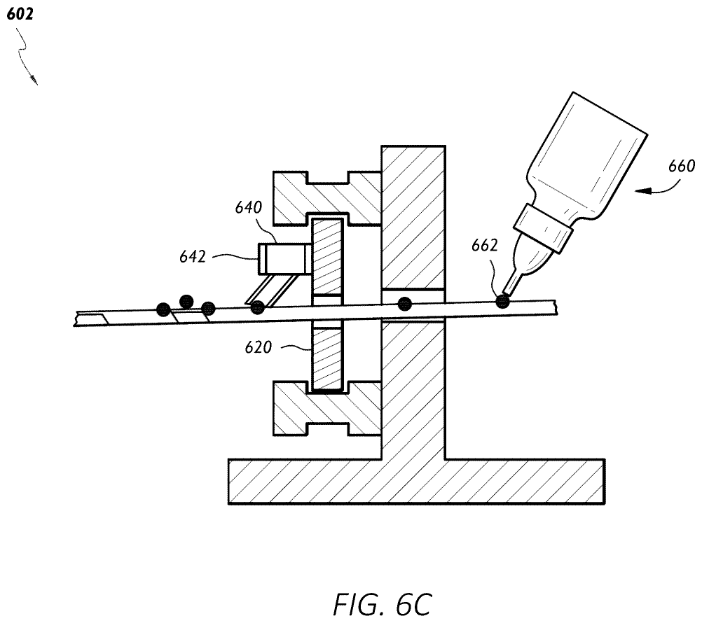

FIGS. 6A and 6B show an example system 600 for helixing electrical wires 642 (also referred to more generally as a “payload”) around a working channel 601 (also referred to more generally as a “target”), according to some implementations. More specifically, FIG. 6A shows a front view of the system 600 and FIG. 6B shows a side view of the system 600. In some implementations, the electrical wires 642 and the working channel 601 may be examples of the electrical wires 502 and the working channel 510, respectively, of FIGS. 5A and 5B. Although described in the context of helixing electrical wires around a working channel, those of skill in the art will recognize that the example system 600 can be generally used for wrapping or helixing any payload 642 around any target 601 having the same or similar properties as the electrical wires and the working channel described in the present disclosure. For example, in some other implementations, the target 601 may be an endoscope. More generally, the terms “target” or “working channel,” as used herein, may refer to any instrument with or without an inner lumen (to allow EM tracking of catheters or tools in other applications such as circulatory or cardiac catheterization).

The system 600 includes a rigid stand 610 with a teardrop-shaped cutout on the face of the stand so that the hole in the center of the cutout can be accessed from an opening in the side. A motorized plate 620 in the shape of a “C” is coupled to the stand 610, overlapping the cutout. The motorized plate 620 has a similar teardrop-shape cutout, where the opening of the “C” can be aligned with the opening of the cutout on the face of the stand 610 so that the target (or working channel) 601 can be inserted into the center hole of both structures 610 and 620. The motorized plate 620 includes a number of spools (or bobbins) 640 and 650 on which various elements can be wrapped. In the example of FIG. 6, the first spool 640 carries the payload (or electrical wires) 642 and the second spool 650 carries a thermoplastic (or static cling) film 652. Although the motorized plate 620 is shown to include two spools 640 and 650 in the example of FIG. 6, in some other implementations the motorized plate 620 may have any number of spools (depending on the number of elements to be wrapped around the target 601).

In operation, a drive wheel 630 spins the motorized plate 620 around its central axis, wrapping the electrical wires 642 and thermoplastic film 652 around the working channel 601. In some implementations, the second spool 650 may lag the first spool 640 in phase so that the electrical wires 642 are wrapped around the working channel 601 first and the thermoplastic film 652 is wrapped over the electrical wires 642, thereby encapsulating the electrical wires 642 onto the working channel 601. A motorized sled or winch (not shown for simplicity) pulls the working channel 601 through the hole as the motorized plate 620 wraps the elements 642 and 652 around the channel 601. The lateral movement of the working channel 601 combined with the spinning of the electrical wires 642 creates the helical design shown in FIG. 5B. The twist rate or frequency of the helixes depends on the speed at which the motorized plate 620 spins and the rate at which the working channel 601 is pulled through the hole. The wrapped portion of the working channel 601 is fed through a hot die or other heating element which reflows the thermoplastic film 652 to create a more uniform layer.

In some implementations, a controller for the system 600 (not shown for simplicity) may vary the speed at which the motorized plate 620 spins or the rate at which the working channel 601 is pulled through the hole based on the amount of bend or articulation in the underlying section of the working channel 601. As a result, the twist rate of the helixes may vary across the length of the working channel 601. For example, the twist rate may be higher across sections of the working channel 601 with greater bend angle or articulation and the twist rate may be lower across sections of the working channel 601 with less bend angle or articulation. This may ensure that any compressive and tensile loads balance each other out when the working channel 601 bends or articulates (such as described with reference to FIGS. 5A and 5B).

FIG. 6C show another example system 602 for helixing the electrical wires 642 around the working channel 601, according to some implementations. The system 602 includes the same rigid stand 610 and motorized plate 620 as the system 600 of FIGS. 6A and 6B. However, in the example of FIG. 6C, the motorized plate 620 only includes the first spool 640 which carries the payload (or electrical wires) 642. In other words, the system 602 does not wrap a thermoplastic film around the working channel 601. Instead, the system 602 includes an adhesive element 660 which applies droplets (or streaks) 662 of adhesive (such as a liquid ultraviolet-cure adhesive) onto the surface of the working channel 601 prior to being wrapped with the electrical wires 642. The frequency or timing of the droplets 662 may depend on the twist rate of the helixes so that the droplets 662 are aligned with the electrical wires 642 on the surface of the working channel 601. An ultraviolet wand (not shown for simplicity) may be used to cure the adhesive after the electrical wires 642 are wrapped around the working channel 601, thereby affixing the electrical wires 642 to the outer surface of the working channel 601.

Aspects of the present disclosure further recognize that the helical configuration described with reference to FIGS. 5A and 5B also can be used to affix a sensor load to the inner diameter of an endoscope. Attaching the sensor load to the endoscope in this manner may obviate the need for a separate working channel for certain applications.

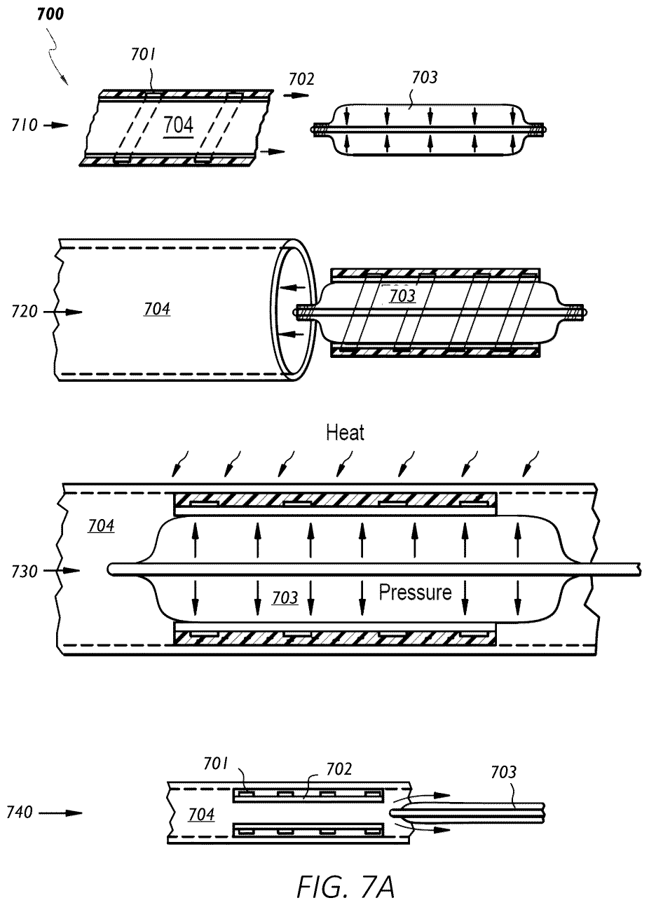

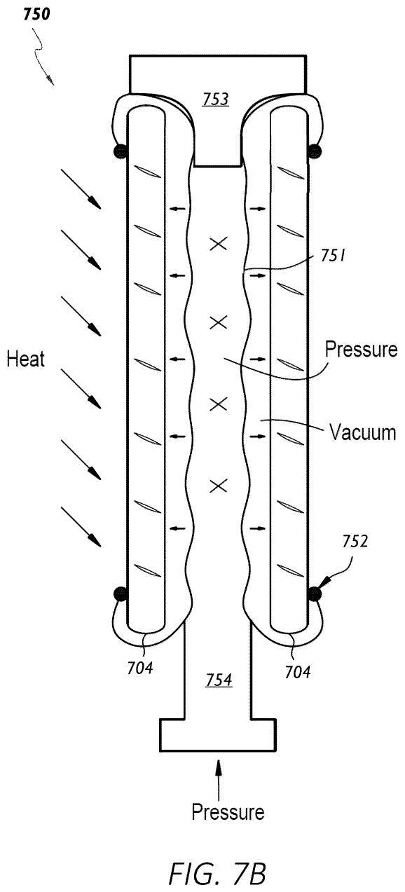

FIG. 7A shows an example system and method 700 for helixing electric wires 701 around an inner diameter of an endoscope 704, according to some implementations. In some implementations, the endoscope 704 may be one example of the endoscope 102 of FIGS. 1-4. The electrical wires 701 may be any discrete or continuous electrical or sensory payload. In some implementations, the electrical wires 701 may be one example of the electrical wires 502 of FIGS. 5A and 5B. However, rather than wrapping the electrical wires 701 around an outer diameter of a working channel (such as described with reference to FIGS. 5-6C), the method 700 attaches the wires to the inner surface of the endoscope 704 in which a working channel may be inserted.

At step 710, the electric wires (or payload) 701 are coated with a thermoplastic film (such as the thermoplastic film 652 of FIGS. 6A and 6B) 702 and attached to an outer surface of a balloon 703. Alternatively, or in addition, the electrical wires 701 may be coated with an adhesive that can be activated by heat or pressure (such as the adhesive 662 of FIG. 6C). In some implementations, the balloon 703 may be in a “collapsed” or at least semi-deflated state when the electrical wires 701 are attached thereto.

At step 720, the balloon 730 is inserted into the inner diameter of the endoscope 704 with the electrical wires 701 attached thereto. In some implementations, the balloon 703 may still be in the collapsed state when inserted into the endoscope 704.

At step 730, the balloon 730 is expanded under high heat to reflow the thermoplastic layer 702 (or activate the adhesive). In some implementations, step 730 may be performed during a lamination step in the process of manufacturing the endoscope 704.

At step 740, after the endoscope 704 has cooled, the balloon 730 is deflated and removed from the endoscope 704, leaving behind the electrical wires 701 affixed to the inner diameter of the endoscope 704. For example, the electrical wires 701 adhere to the endoscope 704 via the thermoplastic film 702 (or the adhesive).

FIG. 7B shows another example system and method 750 for helixing electric wires around an inner diameter of the endoscope 704, according to some implementations. In the example of FIG. 7B, the electric wires are combined with the thermoplastic film to produce a “plastic layer” 751 that can be inserted into the inner diameter of the endoscope 704. The plastic layer 751 is held in place via tacks 752 on the outer diameter of the endoscope 704. With the plastic layer 751 held in place by the tacks 752, one end of the endoscope 704 is sealed using a plug 753 while the other end of the endoscope 704 may be sealed using a valve 754 that can create or introduce pressure inside the endoscope 704. The pressure pushes the plastic layer 751 against the inner surface of the endoscope 704, creating a vacuum seal. While the plastic layer 751 is vacuum sealed against the inner surface of the endoscope 704, the endoscope 704 may be heated to reflow the thermoplastic film. After the endoscope 704 has cooled, the plug 753 and the valve 754, as well as the tacks 752, can be removed from the endoscope 704, thereby leaving behind the plastic layer 751 adhered to the inner diameter of the endoscope 704.

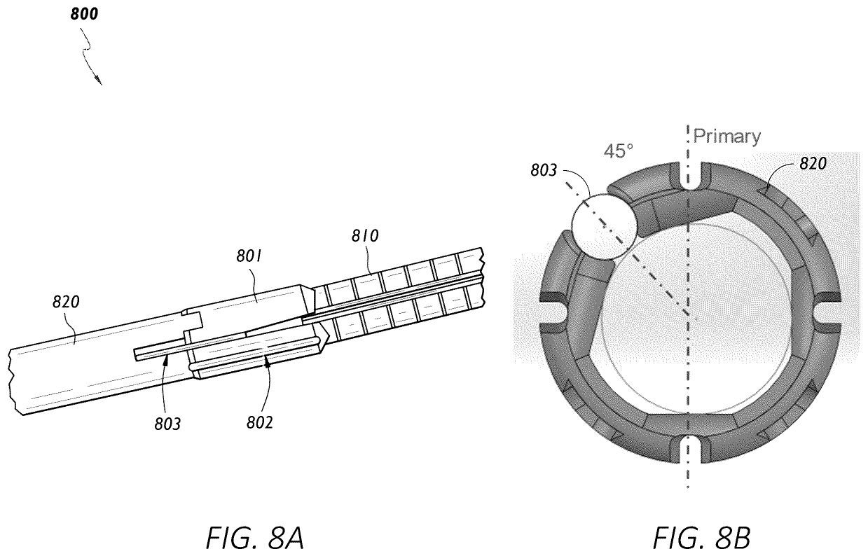

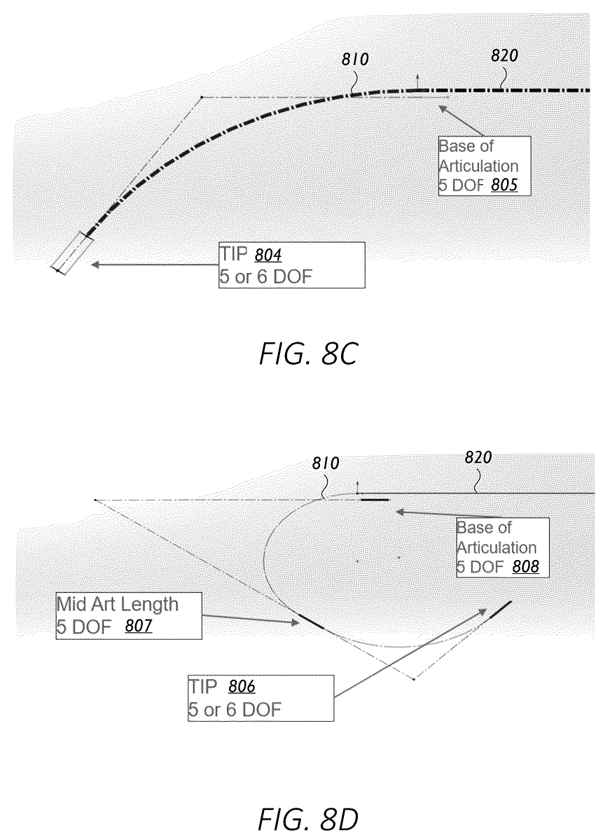

FIGS. 8A-8D show an example endoscope 800 having sensor elements 802 integrated into its walls, according to some implementations. More specifically, FIG. 8A shows a side view of the endoscope 800, FIG. 8B shows a cross-section of the endoscope 800, and FIGS. 8C and 8D show example articulations of the endoscope 800. In some implementations, the endoscope 800 may be one example of the scope 102 of FIGS. 1-4. The endoscope 800 includes an articulating section 810 and a passive section 820 (also referred to as a “flexure”) coupled together via a mechanical coupler 801. The sensor elements 802 may include any types of sensor elements that can be coupled to a working channel. In some implementations, the sensor elements 802 may include EM sensors that can be used for determining a pose of the endoscope 800.

In some implementations, one or more of the sensor elements 802 can be at least partially disposed or embedded within a pocket 803 or cutout of the passive section 820 and the mechanical coupler 801 at the base of the articulating section 810. As described with reference to FIGS. 5A and 5B, the articulating section 810 may be articulated via one or more articulation mechanisms (such as the tendons 503 of FIG. 5A) to bend or deflect at desired angles. By contrast, the passive section 820 merely bends or follows the articulation of the articulating section 810. Thus, to support accurate positioning of the endoscope 800, the sensor elements 802 may be mechanically coupled to the articulation mechanism (such as via links or flexures). The sensor elements 802 can be coupled or attached to the endoscope 800 using any suitable means (including adhesives, laser welding, or other mechanical means). For example, in some implementations, one or more of the sensor elements 802 may be discrete sensors that are directly mounted to the endoscope 800. In such implementations, each discrete sensor may be coupled to a respective wire such that multiple wires are bundled together to form a sensor load (such as the sensor load 502 of FIGS. 5A and 5B). In some other implementations, one or more of the sensor elements 802 may be coupled to a flexible printed circuit board (PCB) that is mounted to the endoscope 800. In such implementations, each PCB may be coupled to one or more wires that form at least part of the sensor load.

As shown in FIGS. 8C and 8D, a single EM sensor having 5 degrees of freedom may be insufficient to establish the orientation of the endoscope 800. Thus, in some implementations, one or more additional sensors (similar, if not identical, to the sensor elements 802) may be embedded elsewhere on the endoscope 800. FIG. 8C shows an example articulation of the endoscope 800 that can be measured using a 2-sensor configuration, where the sensors are embedded in the distal tip (804) and the base (805) of the articulating section 810. FIG. 8D shows another example articulation of the endoscope 800 that can be measured using a 3-sensor configuration, where the sensors are embedded in the distal tip (806), the midpoint (807), and the base (808) of the articulating section 810. The number of sensors and/or spacing between the sensors may depend on various factors including, but not limited to, the degree of bend or articulation of the endoscope 800, the twist rate of the sensor load, and/or a desired level of granularity for which the shape or orientation of the endoscope 800 is to be measured. For example, the sensors 806-808 may or may not be equidistantly spaced in some implementations.

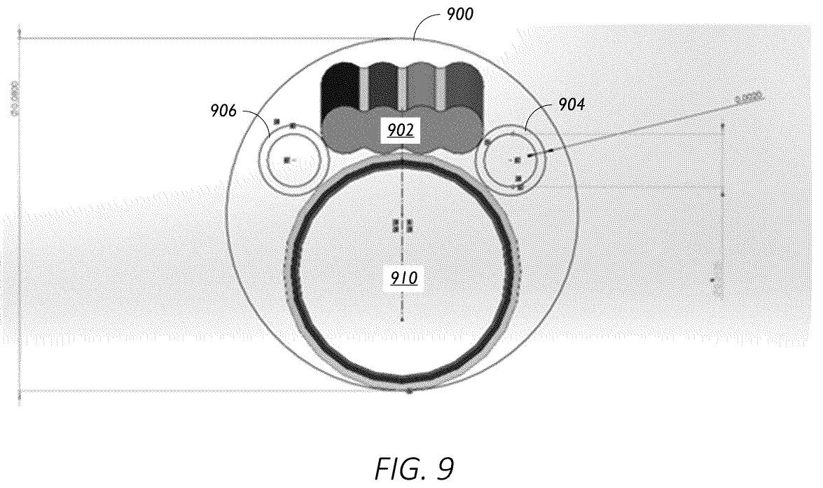

FIG. 9 shows a cross-section of an endoscope 900, according to some implementations. In some implementations, the endoscope 900 may be one example of the scope 102 of FIGS. 1-4. Disposed within an inner diameter of the endoscope 900 is a working channel 910, electrical wires (or sensor load) 902, and a number of micro-fluidics lumens 904 and 906. Although 4 electrical wires 902 and 2 micro-fluidics lumens 904 and 906 are depicted in the example of FIG. 9, in actual implementations the working channel 910 may be coupled to any number of electrical wires and any number of micro-fluidics lumens.

The micro-fluidics lumens 904 and 906 are hollow tubes that are attached or coupled to the electrical wires 902 and provide additional modalities or features for the working channel 910. For example, in some implementations, one or more of the micro-fluidics lumens 904 and 906 may be coupled to a pressure sensor that can measure the pressure inside a patient's organ based on the air pressure inside the lumen. In some other implementations, the micro-fluidics lumens 904 and 906 may be used for irrigation and aspiration. For example, while navigating the working channel 910 within a patient's organ (such as a lung), a camera or other sensor at the tip of the working channel 910 may become covered with an opaque substance (such as phlegm) that occludes the camera. In some implementations, a first micro-fluidic lumen 904 may be used for irrigation, for example, to clear the opaque substance from the working channel 910. In some implementations, the second micro-fluidic lumen 906 may be used for aspiration, for example, to remove or extract the opaque substance from the organ. Still further, in some implementations, the micro-fluidics lumens 904 and 906 can be used to transport other elements or materials in addition to, or in lieu of, fluid or air. Example suitable transport elements may include, but are not limited to, sensitive solid or electrical elements which require physical, optical, or electrical isolation from other elements within the main lumen of the endoscope.

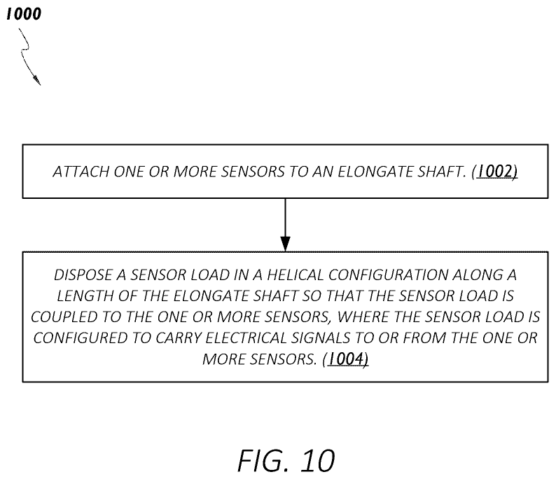

FIG. 10 shows an illustrative flowchart depicting an example operation 1000 for assembling a medical instrument, according to some implementations. In some implementations, the example operation 1000 may be performed by an instrument assembly system, such as any of the systems 600, 602, 700, and/or 750 of FIGS. 6A-7B.

The system attaches one or more sensors to an elongate shaft (1002). The system further disposes a sensor load in a helical configuration along a length of the elongate shaft so that the sensor load is coupled to the one or more sensors, where the sensor load is configured to carry electrical signals to or from the one or more sensors (1004). In some aspects, the sensor load may be wrapped around an outer diameter of the elongate shaft. In such aspects, the elongate shaft may comprise a working channel. In some other aspects, the sensor load may be disposed on an inner diameter of the elongate shaft.

In some implementations, the disposing of the sensor load on the inner diameter of the elongate shaft may include attaching the sensor load to an outer surface of a balloon, inserting the balloon with the sensor load attached thereto into the inner diameter of the elongate shaft, expanding the balloon so that the sensor load is pressed against the inner surface of the elongate shaft, and affixing the sensor load to the inner surface of the elongate shaft while the balloon is expanded. In some other implementations, the sensor load may be vacuum sealed to the inner diameter of the elongate shaft.

In some aspects, the disposing of the sensor load in the helical configuration may include adjusting a twist rate of the helical configuration based on a degree of bend by which the elongate shaft is articulable. In some implementations, the twist rate may vary along the length of the elongate shaft.

In some implementations, the disposing of the sensor load along the length of the elongate shaft may include affixing the sensor load to the elongate shaft using a thermoplastic film. In some other implementations, the disposing of the sensor load along the length of the elongate shaft may include affixing the sensor load to the elongate shaft using an adhesive.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A medical instrument comprising:
an elongate shaft;
one or more sensors coupled to the elongate shaft; and
a sensor load coupled to the one or more sensors and disposed in a helical configuration along a length of the elongate shaft, the sensor load being configured to carry electrical signals to or from the one or more sensors.

2. The medical instrument of claim 1, wherein the sensor load is disposed on an outer diameter of the elongate shaft.

3. The medical instrument of claim 2, wherein the elongate shaft comprises a working channel.

4. The medical instrument of claim 1, wherein the sensor load is disposed on an inner diameter of the elongate shaft.

5. The medical instrument of claim 4, wherein the elongate shaft comprises an endoscope.

6. The medical instrument of claim 1, wherein the helical configuration has a twist rate associated with a degree of bend by which the elongate shaft is articulable.

7. The medical instrument of claim 6, wherein the twist rate is configured to balance tensile and compressive forces on the sensor load in response to articulation of the elongate shaft.

8. The medical instrument of claim 6, wherein the twist rate varies along the length of the elongate shaft.

9. The medical instrument of claim 1, wherein the one or more sensors include a first sensor coupled to a distal tip of the elongate shaft and a second sensor coupled to a base of the elongate shaft.

10. The medical instrument of claim 9, wherein the one or more sensors further include a third sensor coupled to a midpoint of the elongate shaft.

11. The medical instrument of claim 1, further comprising:
one or more micro-fluidics lumens coupled to the sensor load and disposed along the length of the elongate shaft.

12. A method for assembling a medical instrument, comprising:
attaching one or more sensors to an elongate shaft; and
disposing a sensor load in a helical configuration along a length of the elongate shaft so that the sensor load is coupled to the one or more sensors, the sensor load being configured to carry electrical signals to or from the one or more sensors.

13. The method of claim 12, wherein the sensor load is wrapped around an outer diameter of the elongate shaft.

14. The method of claim 13, wherein the elongate shaft comprises a working channel.

15. The method of claim 12, wherein the sensor load is disposed on an inner diameter of the elongate shaft.

16. The method of claim 15, wherein the disposing of the sensor load on the inner diameter of the elongate shaft comprises:
attaching the sensor load to an outer surface of a balloon;
inserting the balloon with the sensor load attached thereto into the inner diameter of the elongate shaft;
expanding the balloon so that the sensor load is pressed against an inner surface of the elongate shaft; and
affixing the sensor load to the inner surface of the elongate shaft while the balloon is expanded.

17. The method of claim 15, wherein the sensor load is vacuum sealed to the inner diameter of the elongate shaft.

18. The method of claim 12, wherein the disposing the sensor load in the helical configuration comprises:
adjusting a twist rate of the helical configuration based on a degree of bend by which the elongate shaft is articulable.

19. The method of claim 18, wherein the twist rate varies along the length of the elongate shaft.

20. The method of claim 12, wherein the disposing of the sensor load along the length of the elongate shaft comprises:
affixing the sensor load to the elongate shaft using a thermoplastic film or an adhesive.
